(19) United States
(12) Patent Application Publication
(10) Pub. No.: US 2007/0179181 A1
(43) Pub. Date: Aug. 2, 2007

(54) Treatment of inflammatory disorders and pain using beta-aminoalcohols

(76) Inventors: Andrew Douglas Baxter, Essex (GB); John Brew, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/591,137

(22) PCT Filed: Mar. 17, 2005
(86) PCT No.: PCT/GB05/01031

§ 371 Date: Oct. 6, 2006

(30) Foreign Application Priority Data
Mar. 17, 2004 (GB) 0406016.6
Aug. 19, 2004 (GB) 0418556.7
Oct. 14, 2004 (GB) 0422880.5

(51) Int. Cl. A61K 31/47

(52) U.S. Cl. 514/314

(57) ABSTRACT

A compound that has utility in the treatment or prevention of a condition associated with T-cell proliferation or that is mediated by pro- and/or anti-inflammatory cytokines, is of formula (I) wherein R₁ is H or Me; R₂ is H or alkyl and R₃ is H or Me, or R₂ and R₃ are —CH₂— thereby forming a ring; n is 0 to 2; X is CH₂ or O; and the two benzene rings are each optionally substituted with OH, OMe, Halogen, NHCHO, NHSO₂Me, CONH₂, SOMe OCH₂O or CH₂OH.

### FIELD OF THE INVENTION

This invention relates to the treatment of inflammatory disorders and pain.

### BACKGROUND OF THE INVENTION

Immune-driven inflammatory events are a significant cause of many chronic inflammatory diseases where prolonged inflammation causes tissue destruction and results in extensive damage and eventual failure of the effected organ. The cause of these diseases is unknown, so they are often called autoimmune, as they appear to originate from an individual's immune system turning on itself. These conditions include systemic lupus erythematosus (SLE) and rheumatoid arthritis.

In addition, there are chronic inflammatory diseases whose aetiology is more or less known but whose inflammation is also chronic and unremitting. These also exhibit massive tissue/organ destruction and include conditions such as osteoarthritis. These conditions are a major cause of illness in the developing world and are poorly treated by current therapies.

Inflammation of skin structures (dermatitis) is a common set of conditions. These diseases are treated using a wide array of therapies, many of which have very severe side-effects.

Current disease-modifying treatments (if any) for immune-driven conditions, include neutralising antibodies, cytotoxics, corticosteroids, immunosuppressants, antihistamines and antimuscarinics. These treatments are often associated with inconvenient routes of administration and severe side-effects leading to compliance issues. Moreover, certain drug classes are only effective for certain types of inflammatory diseases, e.g. antihistamines for rhinitis.

Various beta-aminoalcohols are known, including bufeniode, denopamine, fenoterol, formoterol, ifenprodil, isoxsuprine, labetalol, medroxalol, mesuprine, nylidrin, protokylol, ractopamine, ritodrine, salmefamol and sulfinalol. They have antihypertensive, vasodilator, sympathomimetic, bronchodilator or cardiostimulant activity through agonism and antagonism at alpha and beta adrenoceptors. These agents have at least one chiral centre, and their activity at the alpha or beta adrenoceptors resides mainly or solely in one of the enantiomers. If the molecule has more than one chiral centre, the activity at the alpha or beta adrenoceptors resides mainly in one of the diastereomers.

### SUMMMARY OF THE INVENTION

Surprisingly, it has been found that phenyl substituted beta-amino alcohols (I) are inhibitors of cytokines and possess anti-inflammatory properties. According to the present invention, pain or an inflammatory condition, e.g. as described above, is treated by the use of a compound of general formula (I) 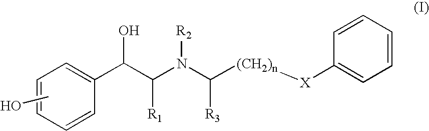
wherein R₁ is H or Me;

R₂ is H or alkyl and R₃ is H or Me, or R₂ and R₃ are —CH₂— thereby forming a ring;

n is 0 to 2;

X is CH₂ or O; and

the two benzene rings are each optionally substituted with OH, OMe, halogen, NHCHO, NHSO₂Me, CONH₂, SOMe, OCH₂O or CH₂O H.

### DESCRIPTION OF PREFERRED EMBODIMENT

Compounds of formula (I) include bufeniode, denopamine, fenoterol, formoterol, ifenprodil, isoxuprine, labetalol, medroxalol, mesuprine, nylidrin, protokylol, ractopamine, ritodrine, salmefamol and sulfinalol. It will be understood that the invention refers to salts, e.g. the hydrochloride, metabolites and pro-drugs thereof, as well as any diastereomers and enantiomers of (I).

A preferred diastereomer or enantiomer of (I) has little or no activity at the αor β adrenoceptors. This activity may be determined vitro assay, e.g. as described above. In particular, it has been found that for beta-amino alcohols (I), the enantiomers or diastereomers that have little or no activity at the α or β adrenoceptors are inhibitors of cytokines and possess anti-inflammatory properties as well as reducing pain in pain conditions where cytokines are involved.

According to one aspect of the present invention, an inflammatory condition, e.g. as previously described, is treated by the use of enantiomers or diastereomers of beta-amino alcohols (I) that have little or no activity at the α or β adrenoceptors. According to another aspect of the invention, pain such as acute, chronic or neuropathic pain (including, but not limited to, pain associated with cancer, surgery, arthritis, dental surgery, painful neuropathies, trauma, musculo-skeletal injury or disease, and visceral diseases) and migraine headache in mammals, can be treated by the use of enantiomers or diastereomers of beta-amino alcohols (I) that have little or no activity at the α or β adrenoceptors.

A compound of formula (I) may be used to treat an inflammatory disease including, but not exclusive to, autoimmune diseases involving multiple organs, such as systemic lupus erythematosus (SLE) and scleroderma, specific tissues or organs such as the musculoskeletal tissue (rheumatoid arthritis and ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy and interstitial nephritis), exocrine glands (Sjogren's syndrome and autoimmune pancreatitis) and skin (psoriasis and atopic dermatitis), chronic inflammatory diseases such as osteoarthritis, periodontal disease, diabetic nephropathy, chronic obstructive pulmonary disease, atherosclerosis, graft versus host disease, chronic pelvic inflammatory disease, endometriosis, chronic hepatitis and tuberculosis and IgE-mediated (Type I) hypersensitivities such as rhinitis, asthma, anaphylaxis and dermatitis. Dermatitis conditions that may be treated include actinic keratosis, acne rosacea, acne vulgaris, allergic contact dermatitis, angioedema, atopic dermatitis, bullous pemiphigoid, cutaneous drug reactions, erythema multiforme, lupus erythrometosus, photodermatitis, psoriasis, psoriatic arthritis, scleroderma and urticaria.

This invention also relates to the treatment of patients (including man and/or mammalian animals raised in the dairy, meat or fur industries or as pets) suffering from chronic, acute or neuropathic pain. Compounds of the invention, and in particular, the preferred enantiomers or diastereomers of compounds of formula (I), can be used among other things in the treatment of pain conditions such as acute and chronic pain (as well as, but not limited to, pain associated with cancer, surgery, arthritis, dental surgery, trauma, musculo-skeletal injury or disease and visceral diseases) and migraine headache. Painful conditions that can be treated also include neuropathic pain (post-herpetic neuralgia, diabetic neuropathy, drug induced neuropathy, HIV mediated neuropathy, sympathetic reflex dystrophy or causalgia, fibromyalgia, myofacial pain, entrapment neuropathy, phantom limb pain, trigeminal neuralgia. Neuropathic conditions include central pain related to stroke, multiple sclerosis, spinal cord injury, arachnoiditis, neoplasms, syringomyelia, Parkinson's and epilepsia.

Any suitable route of administration can be used. For example, any of oral, topical, parenteral, ocular, rectal, vaginal, inhalation, buccal, sublingual and intranasal delivery routes may be suitable. The dose of the active agent will depend on the nature and degree of the condition, the age and condition of the patient and other factors known to those skilled in the art. A typical dose is 10-100 mg given one to three times per day.

It will often be advantageous to use compounds of the invention in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

Compounds may be used according to the invention when the patient is also administered or in combination with another therapeutic agent selected from corticosteroids (examples include cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone), disease modifying anti-rheumatic drugs (DMARDs) (examples include azulfidine, aurothiomalate, bucillamine, chlorambucil, cyclophosphamide, leflunomide, methotrexate, mizoribine, penicillamine and sulphasalazine), immunosuppressants (examples include azathioprine, cyclosporin, mycophenolate), COX inhibitors (examples include aceclofenac, acemetacin, alcofenac, alminoprofen, aloxipirin, amfenac, aminophenazone, antraphenine, aspirin, azapropazone, benorilate, benoxaprofen, benzydamine, butibufen, celecoxib, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, mefenamic acid, metamizole, mofebutazone, mofezolac, nabumetone, naproxen, nifenazone, oxametacin, phenacetin, pipebuzone, pranoprofen, propyphenazone, proquazone, rofecoxib, salicylamide, salsalate, sulindac, suprofen, tiaramide, tinoridine, tolfenamic acid, zomepirac), neutralising antibodies (examples include etanercept and infliximab) and antibiotics (examples include doxycycline and minocycline).

The following studies provide evidence on which the present invention is based.

Alphal Adrenoceptor Binding Affinity

Brains from Wistar rats were homogenised and incubated with test article (over a concentration range) and the radioligand 3H-prazosin (0.25 nM) for 30 minutes at 25° C., in a 50 mM Tris-HCl, 0.1% ascorbic acid, 10 μM pargyline incubation buffer. After washing, binding was measured by scintillation counting. LPS Mouse Assay

7 week old Balb C ByJ mice (24-28 g) were administered, either by i.p. (5 ml/kg) or oral (10 ml/kg) administration, with vehicle or test article. 30 minutes later these animals were challenged with an intraperitoneal injection of 1 mg/kg LPS. 2 hours after LPS challenge blood samples were collected under light isoflurane anaesthesia into normal tubes by retro-orbital puncture. Samples were allowed to clot at room temperature and then spun at 6000 g for 3 min at 4° C. Serum was stored at −20° C. until use. Serum TNFa and IL-10 levels were analysed in duplicate by ELISA technique.

Carrageenan Paw Assay

Fasted (18 hour) male Wistar rats (105-130 g) were weighed and a basal mercury plethysmometer reading was taken of the right hind paw by submerging the paw in the mercury up to the tibiotarsal joint. Subsequently, vehicles, reference items and test articles were administered by oral gavage (10 ml/kg). 30 minutes after treatment, 0.1 ml of 2% carrageenan in 0.9% saline was injected into the subplanatar area of the right hind paw. The right paw was measured again with the plethysmometer, at 1, 2, 3, 4 and 5 hours after carrageenan administration.

Carrageenan Pleurisy Assay

Male mice (˜20 g) were treated orally (10 ml/kg) with test article. After 1 hour, under light isoflurane anaesthesia, the mice were given 1% carrageenan (in 0.9% saline) injected into the pleural cavity. After 3 hours pleural exudate was withdrawn and analysed for volume and peripheral mononuclear cell number. TNFα and IL-10 cytokine levels were then analysed by ELISA.

Rat Adjuvant Assay

Male Wistar rats (180 to 200 g) were inoculated by subplantar injection of freund's adjuvant (suspension of Mycobacterium butyricum in mineral oil) into the right paw at day 0. Sham inoculations were injected in the same way with 0.9% saline in matched Male Wistar rats. On day 2 animals were weighed. On days 3, 4, 7, 9 and 11 animals were weighed and both their right and left hind paws were measure by plethsymometry by submerging the paw up to the tibiotarsal joint. On day 11, rats with left hind paw volumes increased by 20% were selected for continuance in the study. On the same day continuance rats were administered test article orally (10 ml/kg in distilled water) and from then on once a day until the completion of the study. Left and right hind paw volumes were measured on days 11, 14, 15, 16, 18 and 21.

Ifenprodil

The receptor binding affinities at the alpha1 adrenoceptor have been determined for all four enantiomers. These values can be used to estimate composite binding affinities for the two racemate pairs, erythro and threo.

| | |
|---|---|
| Ifenprodil enantiomer | Alpha1 affinity |

| | | |
|---|---|---|
| erythro | (+) | 63 nM |
| erythro | (−) | 482 nM |
| threo | (+) | 2160 nM |
| threo | (−) | 439 nM |

In the LPS mouse assay the two racemates of ifenprodil have identical effects on TNFα levels and very similar effects on IL-10.

Alphal adrenoceptor antagonism is known to raise cAMP levels. cAMP levels are known to modulate cytokine release. Consequently there is a possibility that some of the cytokine modulatory activity exhibited by the erythro racemate is due to its known alpha adrenoceptor antagonism. However, the α½ adrenoceptor receptor binding affinity for the racemate strongly suggest that if this is the predominant mechanism for the cytokine modulatory activity of the two racemates, the TNFα and IL-10 effects would be quite different. Since they are in fact very similar and there are no statistical differences between the effects of the two racemates, it may be concluded that some other mechanism that is shared by the two racemates is responsible for the cytokine modulatory profile observed.

Ritodrine The tocolytic compound ritodrine has been found to have cytokine modulatory activity in terms of the LPS-induced systemic TNFα release in mouse blood. This translates to a functional anti-inflammatory activity described in the carrageenan paw oedema assay; ritodrine (30 mg/kg oral) has a greater effect than ibuprofen (100 g/kg oral).

Labetalol

In the rat adjuvant model of arthritis, two doses (30 mg/kg and 100 mg/kg) of labetalol were tested. Pronounced (and similar) efficacy was observed for both doses.

1. A compound for the treatment or prevention of a condition associated with T-cell proliferation or that is mediated by pro- and/or anti-inflammatory cytokines, wherein the compound is of formula (I) 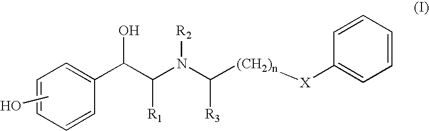
wherein R₁ is H or Me;
R₂ is H or alkyl and R₃ is H or Me, or R₂ and R₃ are —CH₂— thereby forming a ring;
n is 0 to 2;
X is CH₂ or O; and
the two benzene rings are each optionally substituted with OH, OMe, halogen, NHCHO, NHSO₂Me, CONH₂, SOMe, OCH₂O or CH₂OH.

2. The method according to claim 1, wherein the condition is a chronic degenerative disease such as rheumatoid arthritis, osteoarthritis or osteoporosis.

3. The method according to claim 1, wherein the condition is a chronic demyelinating disease such as multiple sclerosis.

4. The method according to claim 1, wherein the condition is a respiratory disease such as asthma or chronic obstructive pulmonary disease.

5. The method according to claim 1, wherein the condition is an inflammatory bowel disease (IBD) such as ulcerative colitis or Crohn's disease.

6. The method according to claim 1, wherein the condition is a dermatological condition such as psoriasis, scleroderma or atopic dermatitis.

7. The method according to claim 1, wherein the condition is a dental disease such as periodontal disease or gingivitis.

8. The method according to claim 1, wherein the condition is diabetic nephrophathy, lupus nephritis, IgA nephrophathy or glomerulonephritis.

9. The method according to claim 1, wherein the condition is systemic lupus erythomatosus (SLE).

10. The method according to claim 1, wherein the condition is graft vs host disease.

11. The method according to claim 1, wherein the condition is a pain condition.

12. The method according to claim 11, wherein the pain condition is chronic pain such as chronic back pain, malignant pain, chronic headache (including migraine and cluster headaches) or arthritic pain).

13. The method according to claim 11, wherein the pain condition is acute pain such as post-operative pain, post-traumatic pain or acute disease-induced pain.

14. The method according to claim 11, wherein the pain condition is neuropathic pain.

15. The method according to claim 1, wherein the compound is selected from bufeniode, denopamine, fenoterol, ifenprodil, isoxsuprine, labetalol, medroxalol, mesuprine, nylidrin, protokylol, ractopamine, ritodrine, salmefamol and sulfinalol.

16. The method according to claim 1, wherein the compound is chiral and is in the form of the enantiomer or diastereomer that has relatively little or no activity at the α or β adrenoceptor.

17. The method according to claim 1, wherein the patient to be treated is also administered another therapeutic agent selected from corticosteroids, cytotoxics, antibiotics, immunosupressants, non-steroidal anti-inflammatory drugs, narcotic analgesics, local anaesthetics, NMDA antagonists, neuroleptics, anti-convulsants, anti-spasmodics, anti-depressants and muscle relaxants.
